(19) United States
(12) Patent Application Publication
(10) Pub. No.: US 2022/0296888 A1
(43) Pub. Date: Sep. 22, 2022

(54) Implantable Electrode Lead with Conductors Connected to Form a Braid

(71) Applicant: BIOTRONIK SE & Co. KG, Berlin (DE)

(72) Inventor: Jan Helge Richter, Berlin (DE)

(73) Assignee: BIOTRONIK SE & Co. KG, Berlin (DE)

(21) Appl. No.: 17/618,097

(22) PCT Filed: Jun. 24, 2020
(86) PCT No.: PCT/EP2020/067602

§ 371 Date: Dec. 10, 2021

(30) Foreign Application Priority Data
Jun. 28, 2019 (EP) 19183189.0

(51) Int. Cl. A61N 1/08; A61N 1/05

(52) U.S. Cl. CPC A61N 1/086; A61N 1/05

(57) ABSTRACT

An implantable electrode lead comprises at least one electrode pole and a plurality of electrical conductors, at least one of which is electrically connected to the at least one electrode pole. The plurality of conductors are connected to one another to form a braid extending along a longitudinal axis, at least one first conductor of the plurality of conductors being helically wound about the longitudinal axis in a first direction of rotation, and at least one second conductor of the plurality of conductors being helically wound about the longitudinal axis in a second direction of rotation, which is opposite the first direction of rotation.

### CROSS-REFERENCE TO RELATED APPLICATIONS

This application is the United States national phase under 35 U.S.C. § 371 of PCT International Patent Application No. PCT/EP2020/067602, filed on Jun. 24, 2020, which claims the benefit of European Patent Application No. 19183189.0, filed on Jun. 28, 2019, the disclosures of which are hereby incorporated by reference herein in their entireties.

### TECHNICAL FIELD

The present invention relates to an implantable electrode lead according to the claims and to a method for producing an implantable electrode lead.

### BACKGROUND

An implantable electrode lead of this kind may be connected to an active electrical device, for example a pacemaker or neurostimulator, and may be implanted, for example, as a cardiac electrode lead in the heart or as a neuroelectrode lead in the spinal cord or even in the brain of a patient. Electrical signals for stimulation may be delivered to the patient via such an electrode lead and an active device connected thereto.

An implantable electrode lead of this kind comprises at least one electrode pole and a plurality of electrical conductors, at least one of which is electrically connected to the at least one electrode pole.

Such an electrode lead is designed to remain in the patient's body usually for a relatively long period of time after implantation. Such an electrode lead is intended to permit examinations on the patient, in particular MRI (magnetic resonance imaging) examinations, which means that an electromagnetic field generated during an MRI examination must not lead to a heating at a conductor or an electrode pole of the electrode lead that might be harmful to the patient.

Heating at an electrode lead implanted in a patient may be caused under certain circumstances by a coupling-in of electromagnetic fields. The coupling of the electrode lead to the electromagnetic field of an MR tomograph (which generates an excitation field with an excitation frequency dependent on the magnetic field strength; at 1.5 Tesla, for example, approximately 64 MHz) is dependent on the effective lead length of conductors of the electrode lead serving, for example, as feed lines for electrode poles. If the effective line length of the electrode lead is in the range of a (series) resonance frequency of the electromagnetic field, electromagnetic fields may be couple into the electrode lead and cause a heating at the electrode lead, which should be avoided if possible.

An electrode lead should generally be thin, especially for neurostimulation. There are also is specifications regarding the length of the feed lines and the maximum ohmic resistance.

An implantable electrode lead is known from U.S. Publication No. 2009/0259281 A1, which has a first, inner conductor and an outer conductor extending outside the inner conductor.

In an electrode device known from U.S. Publication No. 2015/0170792 A1, conductors are arranged helically. In this case, an inner conductor is electrically connected to an electrode pole.

In an electrode lead known from U.S. Publication No. 2008/0147155 A1, electrical conductors comprising polymer threads are connected to one another to form a braid. The electrical conductors in this case are wound helically in a common direction of rotation.

In an electrode lead known from U.S. Publication No. 2009/0099441 A1, conductors in an initial state are interwoven with biodegradable fibers that dissolve when the electrode lead is implanted.

In general, the effective electrical length of an electrical conductor of an electrode lead may be changed by what are known as spur lines, in such a way that electromagnetic energy may no longer be able to couple effectively into the electrode lead at a specific MR excitation frequency, and thus excessive heating does not occur at an electrical conductor of the electrode lead during an MR examination. It should be noted, however, that such spur lines require space to change the effective electrical length of one or more electrical conductors, and this might not be readily available within an electrode lead.

There is a general need for an electrode lead that is easy to manufacture in terms of design and may be used in a versatile manner with regard to the course of electrical conductors in or on the electrode lead and also with regard to MRI compatibility.

The present disclosure is directed toward overcoming one or more of the above-mentioned problems, though not necessarily limited to embodiments that do.

### SUMMARY

An object of the present invention is to provide an implantable electrode lead as well as a method for producing an implantable electrode lead, which in a simple constructional manner enable the installation of electrical conductors in a space-saving manner and flexible adaptability with regard to MRI compatibility.

At least this object is achieved by an object having the features of claim 1.

Accordingly, in the implantable electrode lead, the plurality of conductors are interconnected to form a braid extending along a longitudinal axis, wherein at least one first conductor of the plurality of conductors is helically wound about the longitudinal axis in a first direction of rotation and at least one second conductor of the plurality of conductors is helically wound about the longitudinal axis in a second direction of rotation, which is opposite the first direction of rotation.

The electrode lead has a plurality of electrical conductors that together form a braid that extends along the electrode lead, for example along an inner tube of the electrode lead. For example, each electrical conductor has a core and a surrounding electrical insulation, so that the electrical conductor may carry a current, but adjacent conductors are electrically insulated from each other.

The braid may, for example, have a tubular basic form extending along the longitudinal axis, the longitudinal axis corresponding to a central longitudinal axis of the electrode lead and the braid thus extending around a central lumen of the electrode lead. Alternatively, however, the braid may also be extended along an eccentric secondary lumen of the electrode lead and circumferentially about a longitudinal axis associated with this secondary lumen. The braid is a circumferentially closed hollow body that extends along the length of the electrode lead and is formed by the interwoven conductors of the electrode lead. Alternatively, the braid may also be designed as a plait without lumen.

By connecting the conductors to form a braided body, a flexible, bendable conductor strand is created that may be flexibly adapted for use on a specific electrode lead. In principle, any number of conductors may be braided together within the braid. Available braiding machines enable, for example, the simultaneous braiding of several hundred (electrically conductive) wires or (non-conductive) fibers, and conductors may be braided in two layers in one braiding plane or also in a number of planes one above the other.

The use of such a braid enables flexible adaptability of the electrode lead, for example with regard to MRI compatibility. For example, individual conductors may be used to connect an electrode pole situated at a distal end of the electrode lead to an electrical contact element situated at a proximal end of the electrode lead in order to connect the electrode lead to an active device, such as a pacemaker or neurostimulation device. Other conductors, on the other hand, may be used as what are known as spur lines to extend the effective electrical length of conductors used to connect a contact element to an associated electrode pole. In this case the conductors may be contacted with each other in the desired way or may be electrically separated, so that a flexibly configurable conductor arrangement on the electrode lead may be created by adapting the braiding.

In one embodiment, the braid has a defined length, with the majority of conductors extending along the length of the braid. The conductors forming the braid thus have a common, uniform length corresponding to the entire length of the braid used on the electrode lead. Conductors used to connect an electrode pole to an associated contact element thus have basically the same length, which may be advantageous with regard to MRI compatibility.

To form the braid, the conductors are braided together, and the braid may have a constant io mesh width or a variable mesh width over the length of the electrode lead. By selecting the mesh width, the length of the conductors of the electrode lead may be specified in such a way that a coupling-in of electromagnetic energy at a predetermined MR excitation frequency is reduced if possible, and the electrode lead thus has an advantageous MRI compatibility.

In one embodiment, the majority of conductors are arranged on an inner tube and wound around the inner tube. To produce the electrode lead, the conductors, for example, may be braided around a core on which the inner tube is arranged, so that a tubular braid is formed on the inner tube.

The inner tube defines an inner lumen of the electrode lead. The inner tube may be designed here arbitrarily. For example, the inner tube may have a hydrophilic coating. In one embodiment, for example, the inner tube has a multi-layer structure consisting of layers of different materials.

Outwardly, the braid may be surrounded by conductors through an outer tube. For example, the braid of conductors may be overmoulded with a plastics material. Alternatively, an outer sheath may be produced by what is known as a reflow process, in which tube portions are pushed over the braid arranged on the inner tube and are joined together by melting.

The braid is formed from the conductors of the electrode lead, so that the conductors form a braid body that is tubular in its basic form and extends along the length of the electrode lead. The electrical conductors are wound helically here in different directions of rotation about the longitudinal axis along which the braid extends, and are braided together to form a coherent braid body. First conductors extend helically in a first direction of rotation. Second conductors, on the other hand, extend helically in a second direction of rotation, which is opposite the first direction of rotation, the conductors lying alternately one above the other and one below the other, thus forming the coherent braid.

In one embodiment, the at least one first conductor, which extends in the first direction of rotation about the longitudinal axis, is electrically connected to the at least one electrode pole at a first connection point. Such a conductor thus represents an electrical feed line for the electrode pole.

At a second connection point, in one embodiment, the at least one first conductor is connected, by contrast, to at least one second conductor. The second conductor, which is wound about the longitudinal axis in the opposite direction of rotation to the first conductor in the second direction of rotation, may in this way realize a spur line for extending the effective electrical length of the associated first conductor, so that the effective electrical length of the first conductor serving as a feed line may be adapted so that a coupling-in of electromagnetic energy at a predetermined MR excitation frequency is reduced and excessive heating at the electrode lead during an MRI examination is thereby prevented.

By electrically connecting a first conductor serving as a feed line to a second conductor serving as a spur line, the electrical length of the feed line may be doubled, it also being possible to connect the second conductor in turn to a first conductor, which also serves as a spur line and thus enables an additional extension of the electrical length of the feed line.

In principle, the coupling-in of an electromagnetic field at a predetermined MR excitation frequency—for example, approximately 64 MHz at an MR magnetic field strength of 1.5 Tesla and approximately 128 MHz at an MR magnetic field strength of 3 Tesla—into a conductor is at its maximum at an effective line length corresponding to a series resonance.

At such a series resonance, the magnitude of the impedance is minimal, and due to maximum coupling-in of the electromagnetic field, a field rise and thus a comparatively large heating at the electrode lead may occur. In contrast, if the effective line length of a conductor corresponds to a parallel resonance, the value of the impedance at the conductor is at a maximum, and the coupling-in of the electromagnetic field is suppressed accordingly. It is therefore desirable to set the effective line length of the conductor so that it corresponds to a parallel resonance.

The determination of when parallel resonance occurs at a predetermined MR excitation frequency, for example 64 MHz or 128 MHz, may be carried out by computer simulations or metrologically using suitable test series. For example, the impedance spectrum for different line lengths may be determined metrologically using the reflection coefficient of the conductors when simulating human tissue through a saline solution. This may be used to determine an advantageous effective line length at the predetermined MR excitation frequency, which corresponds to a parallel resonance. Based on this effective line length determined in this way, the length of the spur line for a conductor serving as a feed line may then be selected so that the sum of the spur line length and the length of the feed line corresponds to the desired effective line length.

The effective line length may be adjusted here to a first parallel resonance in the impedance spectrum. However, it is also conceivable and possible to adjust the effective line length to a higher-order parallel resonance by extending the spur line length by half a wavelength (or a multiple of half a wavelength).

The conductors of the braid may be connected arbitrarily and separated locally. The braid formed from the conductors may thus be adapted to produce a conductor construction that is especially adapted for favourable MR compatibility. For example, at least one first conductor and/or at least one second conductor may be electrically interrupted at an associated interruption point so that a conductor extending helically about the longitudinal axis is cut at one point.

For example, a laser cutting process may be used to cut a conductor. In an initial state, the braid of conductors is continuous, with each conductor extending along the entire length of the braid and thus having (approximately) the same length as the other conductors. For the configuration of an electrode lead, in particular with regard to MR compatibility at a specific MR excitation frequency, individual conductors may be electrically interconnected and individual conductors may be interrupted so that feed lines for electrode poles may be connected to spur lines to adapt the effective electrical length of the feed lines.

In one embodiment, the at least one electrode pole is annular and extends circumferentially about the longitudinal axis around the braid. The electrode pole may be pushed onto the braid to produce the electrode lead, and the electrode pole may be electrically contacted with an electrical conductor running underneath it, for example by producing a welded or soldered joint, in order to connect the electrode pole to an associated conductor forming a feed line. If there are a plurality of electrode poles, each electrode pole is connected to an associated conductor serving as a feed line, with each conductor serving as a feed line in turn possibly being connected to a further conductor serving as a spur line for adjusting the effective electrical length.

In one embodiment, at least some conductors of the plurality of conductors of the electrode lead are each associated with at least one accompanying fiber, which extends parallel to the particular conductor. The accompanying fiber may be permanently connected to the associated conductor, so that a helically wound line string formed of a conductor and an accompanying fiber is created.

Each conductor may be associated a single accompanying fiber. However, it is also conceivable and possible that a conductor is enclosed between two associated accompanying fibers, wherein (viewed along the longitudinal axis) at each side of conductor there is arranged one accompanying fiber, and this is connected to the conductor, for example.

The accompanying fibers of the conductors are preferably made of an electrically insulating material. However, such accompanying fibers may also be electrically conductive or have an electrically conductive core surrounded by insulation, for example to provide electrical shielding.

In one embodiment, each conductor, measured radially in relation to the longitudinal axis, has a first thickness, while the accompanying fiber associated with the conductor has a second thickness, which is greater than the first thickness. The accompanying fiber is thus thicker than the associated conductor, which causes the accompanying fiber to provide a spacer for the conductor and, in particular, to prevent the conductors of the braid from lying directly against each other and applying pressure to each other. The accompanying fibers may support the individual strands of the braid against each other, thus preventing direct contact between conductors. The accompanying fibers thus provide mechanical protection for the electrical conductors of the electrode lead.

The conductors may be color-coded so that individual conductors of the braid may be distinguished from each other. Additionally or alternatively, accompanying fibers of the conductors may be marked in color so that the individual conductors of the braid may be distinguished from one another via the accompanying fibers.

In one embodiment, the implantable electrode lead has at least one electrical contact element for electrically connecting the implantable electrode lead to an active device. Such an active device, for example, may be designed as a pacemaker, CRT device, defibrillator or also as an electrophysiology device. In this case, the electrode lead—as a cardiac electrode lead—is to be implanted in particular in the heart of a patient. However, the electrode lead may also be used as a neuroelectrode lead for neurostimulation in the spinal cord or brain (what is known as spinal cord stimulation or deep brain stimulation).

In the implanted position, the electrode lead with its electrode poles lies at a stimulation site in the patient, for example in the area of the human heart or in the area of the spinal cord. The active device may also be implanted in the patient as an implantable device (for example in the form of a pacemaker). However, the active device may also be located outside the patient.

While a contact element, for example on a plug of the electrode lead for connection to the active device, is preferably arranged at a proximal end of the electrode lead, an associated electrode pole is usually arranged at a distal end of the electrode lead, to be implanted for example at a stimulation site. The braid formed from the conductors extends from the proximal end of the electrode lead to the distal end, conductors of the braid being electrically connected to associated contact elements in the region of the proximal end and to associated electrode poles in the region of the distal end to form feed lines.

An object is also achieved by a method for producing an implantable electrode lead, comprising the following steps: providing at least one electrode pole; providing a plurality of electrical conductors, at least one of which is to be electrically connected to the at least one electrode pole, the plurality of conductors being interconnected to form a braid extending along a longitudinal axis, at least one first conductor of the plurality of conductors being helically wound about the longitudinal axis in a first direction of rotation and at least one second conductor of the plurality of conductors being helically wound about the longitudinal axis in a second direction of rotation, which is opposite the first direction of rotation; and connecting the at least one electrode pole to at least one conductor of the plurality of conductors.

At least the advantages and advantageous embodiments described above for the electrode lead are also applicable similarly to the method, and therefore reference should also be made to the comments above.

The braid is present, in an initial state, separated from the electrode poles of the electrode lead to be produced and is braided, for example, on an inner tube so that the braid extends around the inner tube of the electrode lead. To connect conductors of the braid to electrode poles, the preferably annularly shaped electrode poles may be pushed onto the braid and connected to an associated electrical conductor of the braid at a predetermined location—defined in particular by a predetermined distance between the electrode poles.

The connection of an electrode pole to an associated electrical conductor, which is to serve as a feed line for the electrode pole, may be produced by a welded or soldered connection, for example.

For example, an electrode pole may have an opening in its annular lateral surface, through which a welded connection may be produced to an electrical conductor situated under the electrode pole. For example, for this purpose—after removing the insulation of the conductor—an edge of the opening may be melted, so that melted material from the electrode pole flows into the area of the opening and electrical contact with the conductor is established.

In addition, however, very different connection methods are possible for the electrical connection of an electrode pole to an associated conductor, for example a laser welding method, a resistance welding method, a soldering method or even a connection by means of clamps.

The braid is preferably formed from conductors of the same length. The conductors thus extend along the entire length of the braid in an initial state and may be electrically connected to associated electrode poles and contact elements and/or to each other to produce the electrode lead. From the braid formed in the initial state from conductors of uniform length, a flexibly adaptable conductor structure may thus be created for the electrode poles for connection to associated contact elements and for adaptation, particularly with regard to MR compatibility.

For this purpose, individual conductors of the braid may also be electrically separated, so that, for example, spur lines of the desired length may be created on feed lines. A conductor may be electrically cut here at one or more interruption points, so that the conductor is electrically interrupted and line portions of shorter length are created.

After configuration of the conductors of the braid, the conductors of the braid are preferably sheathed, with the braid possibly being overmoulded with plastics material for example, or an outer sheath possibly being formed using a reflow process.

To produce an outer sheath by means of a reflow process, for example tube portions may be pushed over the braid arranged on the inner tube in order to then connect these tube portions to each other by melting and thus create a continuous sheath for the electrode lead. The individual portions may have different stiffnesses in one embodiment, so that the electrode lead may be bent in one or more portions in a flexible way, while in other portions it may be made as rigid as possible.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Various concepts underlying the present invention will be explained in greater detail hereinafter with reference to the exemplary embodiments shown in the figures, in which:

FIG. 1 shows a view of an exemplary embodiment of an electrode lead with electrical conductors braided into a braid;

FIG. 2 shows an enlarged sectional view of the detail X according to FIG. 1;

FIG. 3 shows a view of an exemplary embodiment of an electrode lead with a braid of conductors arranged on an inner tube;

FIG. 4 shows a cross-sectional view along the line A-A according to FIG. 3;

FIG. 5 shows a view of another exemplary embodiment of an electrode lead with a braid of conductors arranged on an inner tube;

FIG. 6 shows a cross-sectional view along the line B-B according to FIG. 5;

FIG. 7 shows a view of another exemplary embodiment of an electrode lead with a braid of conductors; and

FIG. 8 shows a schematic view of an electrode lead.

### DETAILED DESCRIPTION

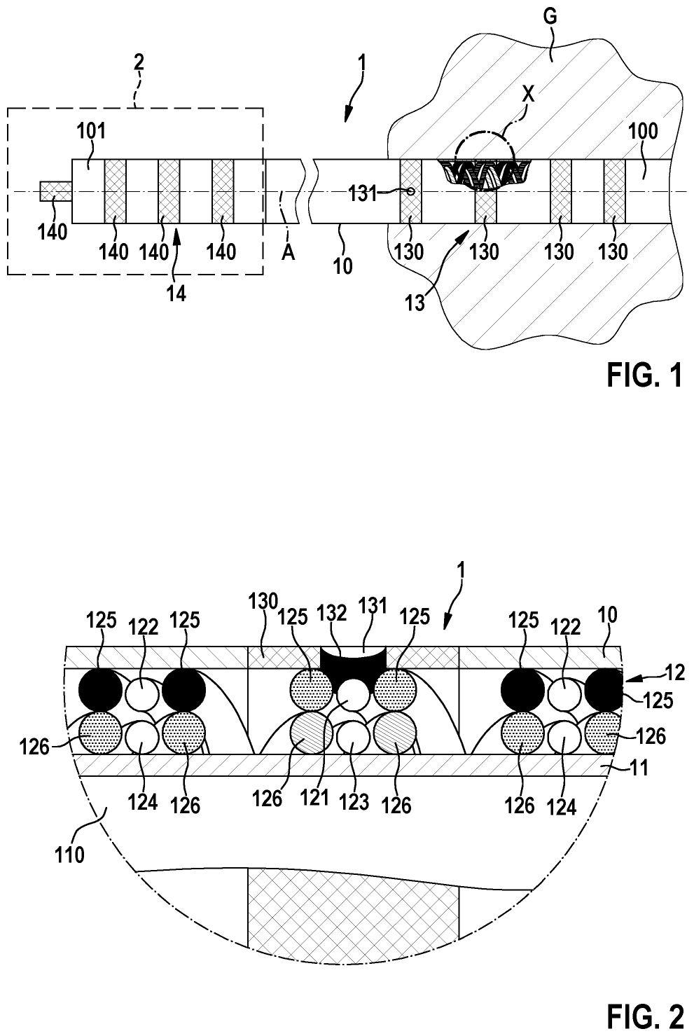

FIG. 1 shows a view of an exemplary embodiment of an electrode lead 1, which is to be connected at a proximal end 101 to an active device 2 and implanted with a distal end 100 in tissue G, for example in the human heart, to effect stimulation at a desired stimulation site, for example.

Such an electrode lead 1 may be used, for example, as a heart electrode lead for implantation in the human heart. Such an electrode lead 1, however, may also be designed as a neuroelectrode lead and thus may be implanted in the spinal cord or brain of a patient.

When used as a cardiac electrode lead, the active device 2 may be designed for example as a pacemaker, CRT device, defibrillator or electrophysiology device, for example for catheter ablation. The active device 2 may also be implanted in one embodiment. Alternatively, the active device 2 may also be operated outside the human body and thus may be connected to the electrode lead 1 outside the human body.

When used as a neuroelectrode lead, the active device 2 is designed for neurostimulation in the spinal cord or human brain (what is known as Spinal Cord Stimulation or Deep Brain Stimulation).

The electrode lead 1 has a plurality of electrode poles 130 arranged in the region of the distal end 100, which electrode poles form an electrode pole arrangement 13 and via which stimulation pulses may be emitted and signals detected. In contrast, a contact arrangement 14 with contact elements 140 is arranged at the proximal end 101 of the electrode lead 1 to form a plug (for example designed according to the IS4/DF4 standard) for electrical connection to an associated active device 2.

Inside an outer tube 10 formed by an outer sheath, electrical conductors are enclosed which serve to electrically connect the contact elements 140 to the electrode poles 130 and for this purpose extend along the length of the electrode lead 1 inside the outer tube 10.

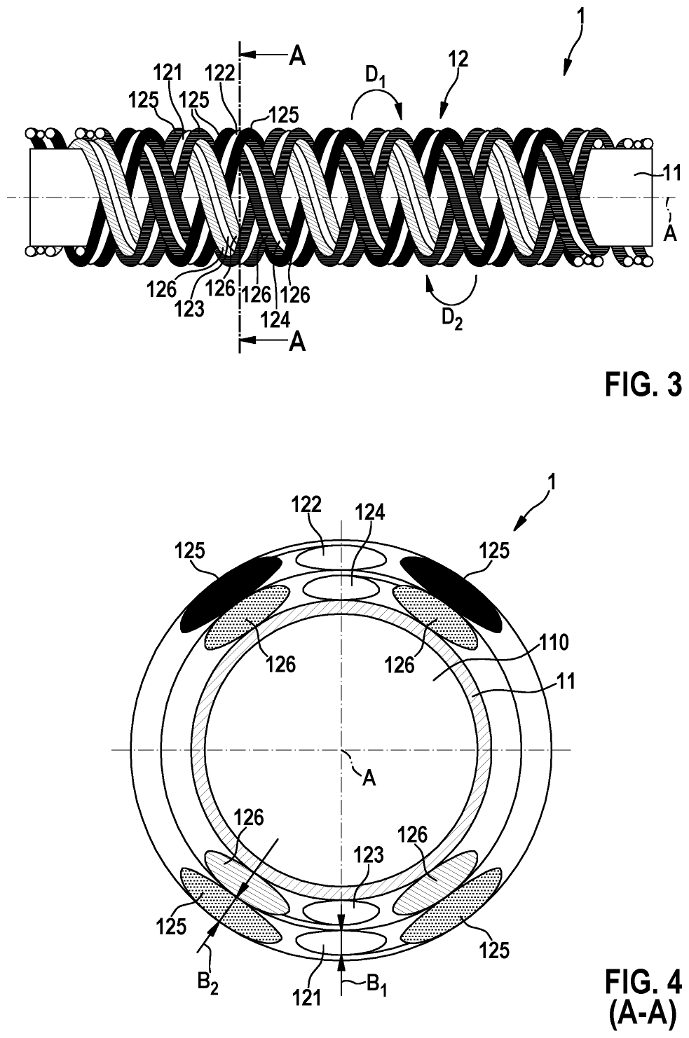

In the electrode lead 1 as per the exemplary embodiment in FIG. 1, electrical conductors 121-124 are interwoven to form a braid 12, as shown in the views in FIGS. 2 to 4. First electrical conductors 121, 122 extend here helically in a first direction of rotation D1 (see FIG. 3) about a longitudinal axis A, along which the electrode lead 1 extends. Second conductors 123, 124, in contrast, are wound helically in a reverse direction of rotation D2 about the longitudinal axis A, with the conductors 123, 124 lying alternately one above the other and one below the other, thus forming a two-layer braid 12 on an inner tube 11 of the electrode lead 1.

The conductors 121-124 of the braid 12 each have an electrically conductive core which is encased by an insulating sheath so that the conductors 121-124 are electrically insulated from each other.

Even if the electrode lead 1 is implanted, medical examinations should be possible without restriction on the patient, in particular also an MRI examination, if necessary even within the scope of the implantation to verify the position of the electrode lead 1. Excessive heating due to a coupling-in of an electromagnetic field within the MRI examination must be avoided in order to exclude injury to a patient.

In the exemplary embodiment shown in FIGS. 1 to 4, a total of four conductors 121-124 are connected together to form a braid 12 and are wound helically around the inner tube 11. In the exemplary embodiment shown, the conductors 121-124 connect the contact elements 140 of the contact arrangement 14 at the proximal end 101 of the electrode lead 1 to the electrode poles 130 of the electrode pole arrangement 13 at the distal end 100 of the electrode lead 1. By selecting the pitch of the helically wound conductors 121-124 and thus by selecting the mesh spacing of the braid 12, the length of the conductors 121-124 may be adjusted so that electromagnetic excitation is effectively prevented at a predetermined MR excitation frequency.

The conductors 121-124 extend along the length of the electrode lead 1 and may preferably have the same length.

In the exemplary embodiment shown, an electrode pole 130, as shown in FIGS. 1 and 2, may be connected to an associated conductor 121 at a specific axial location, for example by producing a welded connection between the electrode pole 130 and the conductor 121. This may be done, for example, by what is known as hole closure welding, during the course of which—after removal of the insulation of conductor 121—an edging of an opening 131 of the electrode pole 130 is melted, and molten material of the electrode pole 130 thereby flows into the area of conductor 121 and thus establishes electrical contact, as may be seen in FIG. 2.

The fact that the electrode poles 130 are annular and the conductors 121-124 for forming the braid 12 extend helically around the inner tube 11 allows an exact axial positioning of the electrode poles 130, in particular in order to set and maintain a predetermined axial distance of the electrode poles 130 from each other. For this purpose, the electrode poles 130 are positioned and twisted on the braiding 12 in such a way that the particular opening 131 of an electrode pole 130 is aligned with an underlying, associated conductor 121-124, and thus a connection to the conductor 121-124 may be produced.

The braid 12 may have further conductors which are not to be connected (directly) to an electrode pole 130 or a contact element 140, but which serve as spur lines to extend the electrical length of the conductors which serve as feed lines and are in contact with the electrode poles 130.

This is shown schematically in FIG. 8. In this way, the braid 12 may be formed from conductors 121, 123 (helically extended, but shown by straight lines in FIG. 8 for reasons of simplified representation), which are wound about the longitudinal axis A of the electrode lead 1 in opposite directions of rotation D1, D2, wherein for example first conductors 121 wound in the first direction of rotation D1 are each electrically contacted at an associated connection point 132 with an associated electrode pole 130, whereas second conductors 123 wound oppositely in the second direction of rotation D2 are each electrically connected at an associated connection point 128 with an associated first conductor 121.

The conductor 121 is connected to the associated electrode pole 130 at the connection point 132 and extends beyond this to the distal end 100 of the electrode lead 1. In the region of the proximal end 101, the conductor 121 is connected to an associated contact element 140, but also extends beyond the contact element 140 to the end of the electrode lead 1. It is not necessary to cut a conductor 121 serving as a feed line, and therefore all conductors 121 serving as feed lines extend over the same length L corresponding to the total length of the electrode lead 1.

In the example shown, the second conductors 123 may be electrically cut at one or more interruption points 127, so that line portions of shorter length are created.

It should be noted that fundamentally different configurations of conductors serving as feed lines and conductors serving as spur lines may be created. In particular, first conductors 121 wound in the first direction of rotation D1 and/or second conductors 123 wound in the second direction of rotation D2 may be used as feed lines, and accordingly second conductors 123 wound in the second direction of rotation D2 and/or first conductors 121 wound in the first direction of rotation D1 may be used as spur lines.

By using conductors 121, 123, which extend along the entire length L of the electrode lead 1, as feed lines or as spur lines, and by electrically connecting a conductor serving as a feed line to another conductor serving as a spur line, the electrical length of a feed line may be doubled, it also being conceivable and possible to connect more than two conductors to each other, so that the effective electrical length of a feed line may also be extended beyond twice the length of the electrode lead 1.

In the exemplary embodiment in FIGS. 1 to 4, each conductor 121-124 is associated with two accompanying fibers 125, 126, which in each case—viewed along the longitudinal axis A of the electrode lead 1—are arranged on both sides of the associated conductor 121-124 and thus enclose the particular conductor 121-124 between them. As may be seen from the sectional view according to FIG. 4, the accompanying fibers 125, 126 each have a thickness B2 (measured radially in cross-section transversely to the longitudinal axis A) which is greater than the thickness B1 of the associated conductor 121-124. This has the effect that the conductors 121-124 are not in direct mechanical contact with each other, but are supported relative to each other via the accompanying fibers 125, 126, which protects io the conductors 121-124 from damage.

The accompanying fibers 125, 126 in each case may be permanently connected to the associated conductors 121-124. However, it is also conceivable and possible to lay the accompanying fibers 125, 126 loosely next to the conductors 121-124.

For production, the inner tube 11 is pushed onto a core that is rigid, for example, and the conductors 121-124 are braided around the inner tube 11 to form the braid 12, for example using a braiding machine. In this case, the accompanying fibers 125, 126 are braided together with the conductors 121-124.

After braiding the braid 12, the individual conductors 121-124 may be electrically connected to associated electrode poles 130 of the electrode pole arrangement 13 and to contact elements 140 of the contact arrangement 14. In addition, individual conductors 121-124 may be contacted with each other to create spur lines for extending the effective electrical length of a feed line. The spur line length may be adjusted as required by cutting individual conductors 121-124.

After configuring the braid 12 for the electrical connection of the electrode poles 130 to the contact elements 140, the outer tube 10 is formed on the braid 12. This may be achieved by overmoulding, for example. Alternatively, a reflow process may be used, within the scope of which tube portions are pushed onto the braid 12 and are connected by melting to form an outer sheath. The electrode poles 130 and the contact elements 140 remain accessible from the outside and are not encapsulated.

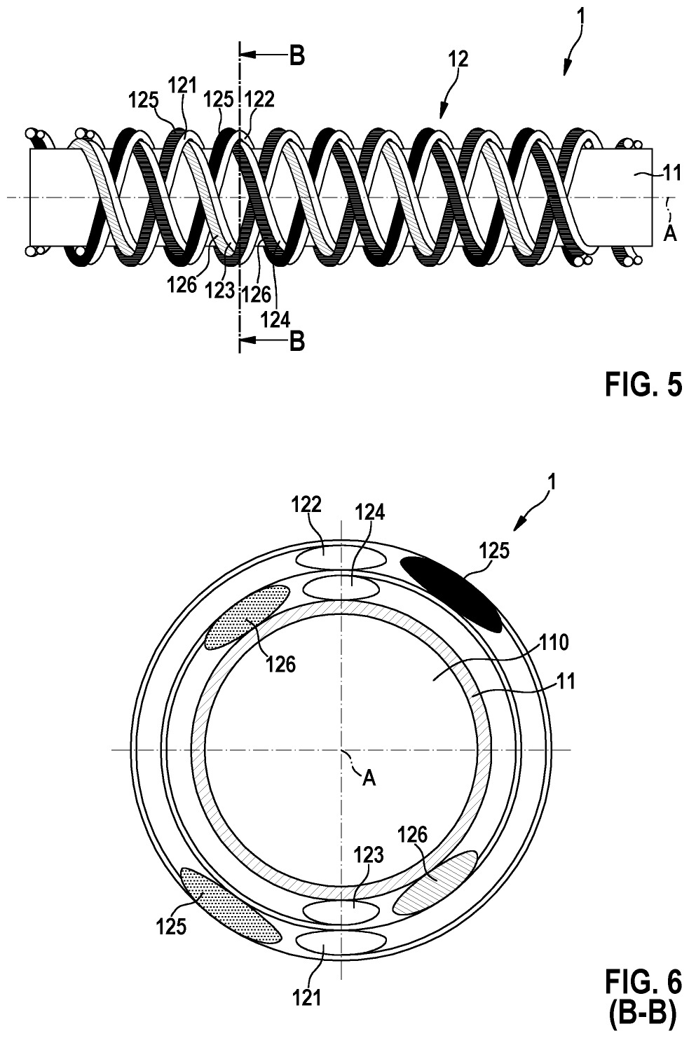

In an exemplary embodiment shown in FIGS. 5 and 6, in comparison to the exemplary embodiment shown in FIGS. 1 to 4, the braid 12 is formed from conductors 121-124, each of which is associated with only one accompanying fiber 125, 126. Otherwise, the exemplary embodiment according to FIGS. 5 and 6 is functionally identical to the exemplary embodiment according to FIGS. 1 to 4, and therefore reference should also be made to the previous comments.

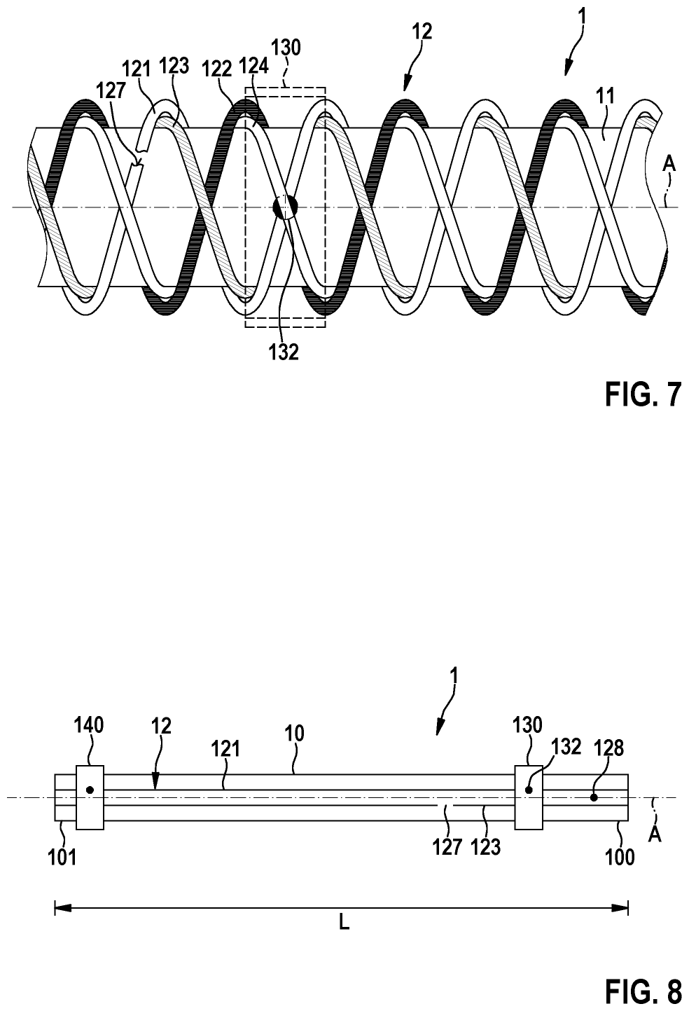

In an exemplary embodiment shown in FIG. 7, conductors 121-124 of the braiding 12 of the electrode lead 1 do not have any accompanying fibers. In FIG. 7, an electrode pole 130 that is to be connected to a conductor 124 as feed line is shown as a dashed line. By contrast, a conductor 121 may serve as a spur line and is electrically cut at an interruption point 127. The conductor 121 may also be contacted with the conductor 124 at a connection point 132, where the electrode pole 130 is electrically contacted with the conductor 124, so that an electrical connection is created between the conductors 121, 124 themselves via the connection point 132 and also between the conductor 124 and the electrode pole 130.

In the exemplary embodiments shown, the conductors 121-124 are interwoven in two layers to form a braid 12, in such a way that the conductors 121-124 extend above and below each other alternately. The braid 12 is thus produced in one braiding plane and (in its basic form) extends in a tubular form about the longitudinal axis A of the electrode lead 1.

It is also conceivable in a different embodiment to form a braid 12 having a plurality of braiding planes, each braiding plane being made in two layers by means of conductors that are extended above and below each other alternately. In this way, the number of conductors of the electrode lead 1 may be increased.

The concepts underlying the present invention are not limited to the exemplary embodiments described above, but may also be realised in other variants.

An electrode lead of the type described here may be used, in principle, in very different applications with associated active devices, for example implantable active devices or also active devices to be used externally of a patient.

The use of a braid formed by conductors of the electrode lead results in a favourable laying of the conductors with good utilisation of the available installation space and flexible configurability of the electrode lead, especially with regard to MRI compatibility.

To produce the braid, multiple conductors may be braided simultaneously in an advantageous manner on an inner tube of the electrode lead, resulting in a tubular basic form which is flexible in its shape and may also be electrically configured by connecting the conductors to electrode poles, contact elements and to each other and by adapting the lengths of the conductors by local cutting.

In principle, the electrode lead may have any number of conductors, for example between two and several hundred conductors, which together form the braid.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE SIGNS

1 implantable electrode

10 outer tube

100, 101 end

11 inner tube

110 lumen

12 braid

121-124 Conductors

125, 126 accompanying fibers

127 interruption point

128 connection point

13 pole arrangement

130 electrode pole

131 opening

132 connection point

14 contact arrangement

140 contact element

2 active device

A longitudinal axis

B1, B2 thickness

D1, D2 direction of rotation

G tissue

L length

1. An implantable electrode lead, comprising,
at least one electrode pole; and
a plurality of electrical conductors, at least one of which is electrically connected to the at least one electrode pole,
wherein the plurality of conductors are connected to one another to form a braid extending along a longitudinal axis, at least one first conductor of the plurality of conductors being helically wound about the longitudinal axis in a first direction of rotation, and at least one second conductor of the plurality of conductors being helically wound about the longitudinal axis in a second direction of rotation, which is opposite the first direction of rotation.

2. The implantable electrode lead according to claim 1, wherein the braid has a length, the plurality of conductors extending along the length of the braid.

3. The implantable electrode lead according to claim 1, wherein the plurality of conductors are arranged on an inner tube and are wound around the inner tube.

4. The implantable electrode lead according to claim 1, wherein the at least one first conductor is electrically connected to the at least one electrode pole at a first connection point and the at least one second conductor is electrically connected to the at least one first conductor at a second connection point.

5. The implantable electrode lead according to claim 1, wherein the at least one first conductor and/or the at least one second conductor has at least one interruption point at which the at least one first conductor and/or the at least one second conductor is electrically interrupted.

6. The implantable electrode lead according to claim 1, wherein the at least one electrode pole is annular and extends circumferentially about the longitudinal axis around the braid.

7. The implantable electrode lead according to claim 1, wherein the at least one electrode pole is electrically connected at a connection point a conductor of the plurality of conductors extending there-beneath.

8. The implantable electrode lead according to claim 1, wherein at least some conductors of the plurality of conductors are each associated with at least one accompanying which extends parallel to the particular conductor.

9. The implantable electrode lead according to claim 8, wherein the accompanying fiber is made of an electrically insulating material.

10. The implantable electrode lead according to claim 8, wherein each conductor, measured radially to the longitudinal axis has a first thickness and the associated at least one accompanying fiber has a second thickness, the second thickness being greater than the first thickness.

11. The implantable electrode lead according to claim 1, further comprising at least one electrical contact element electrically connecting the implantable electrode lead to an active device.

12. A method for producing an implantable electrode lead, comprising the steps of:
providing at least one electrode pole; and
providing a plurality of electrical conductors, at least one of which is to be electrically connected to the at least one electrode pole, the plurality of conductors being interconnected to form a braid extending along a longitudinal axis, at least one first conductor of the plurality of conductors being helically wound about the longitudinal axis in a first direction of rotation and at least one second conductor of the plurality of conductors being helically wound about the longitudinal axis in a second direction of rotation, which is opposite the first direction of rotation; and
connecting the at least one electrode pole to at least one conductor of the plurality of conductors.

13. The method according to claim 12, wherein the at least one electrode pole has an opening, via which the at least one electrode pole is connected to at least one conductor of the plurality of conductors.

14. The method according to claim 12, wherein the braid in an initial state has a length, the plurality of electrical conductors extending along the length of the braid.

15. The method according to 12 wherein at least one conductor of the plurality of conductors is electrically interrupted at an interruption point.
